(19) United States
(12) Patent Application Publication
(10) Pub. No.: US 2021/0047667 A1
(43) Pub. Date: Feb. 18, 2021

(54) METHOD FOR RESTRICTION ENDONUCLEASE-MEDIATED PRIMER GENERATION-ROLLING AMPLIFICATION

(71) Applicant: Ocean University of China, Shandong (CN)

(72) Inventors: Xingguo Liang, Shandong (CN); Ran An, Shandong (CN); Chenru Wang, Shandong (CN); Boying Zhang, Shandong (CN); Shuting Yu, Shandong (CN)

(21) Appl. No.: 16/922,554

(22) Filed: Jul. 7, 2020

(30) Foreign Application Priority Data
Aug. 15, 2019 (CN) 201910752112.5

(51) Int. Cl. C12P 19/34

(52) U.S. Cl. CPC C12P 19/34

(57) ABSTRACT

The present invention relates to a method for restriction endonuclease-mediated primer generation-rolling circle amplification (PG-RCA), including the following steps of: 1) thiol-modifying a single-stranded DNA (ssDNA) at a restriction site of a restriction endonuclease; 2) cyclizing the thiol-modified ssDNA to obtain a thiol-modified circular ssDNA; and 3) using the thiol-modified circular ssDNA obtained in step 2) as a template, adding the template into a reaction system of a target DNA primer to be detected, a DNA polymerase with strand displacement, and a restriction endonuclease for multiple cycles of rolling circle amplification, to amplify a molecular signal of target DNA. The method for RCA provided by the present invention has the following beneficial effects: using a restriction endonuclease to trigger primer generation and establish a restriction endonuclease-mediated rolling circle amplification technique, high amplification efficiency can be guaranteed and the application scope of the technique is broadened.

### TECHNICAL FIELD

The present invention relates to a method for rolling circle amplification (RCA) of DNA, and in particular to a method for triggering primer generation-rolling circle amplification (PG-RCA) using restriction endonuclease-assisted cleavage reactions. The method can achieve rapid and efficient amplification of nucleic acids, and belongs to the technical field of nucleic acid amplification.

### BACKGROUND

Originating in 1990s, RCA, inspired by replication of pathogenic microorganisms in nature, is a process where a circular DNA is used as a template for replication by means of a polymerase with strand displacement, a certain amount of primers, and dNTPs. RCA features no need of complicated heating and cooling processes, and is thus not limited to precision instrument; RCA also features desirable specificity, high sensitivity, and simple operation; therefore, it has been widely used in genome amplification, biology, and medical diagnosis. As RCA technique was investigated and improved unceasingly over the years, a plurality of RCA techniques have been developed.

Common techniques fall into linear RCA and hyperbranched RCA (also known as exponential RCA). However, there are some restrictions. For example, linear RCA has lower efficiency than exponential RCA; exponential RCA has an evident improvement in terms of amplification efficiency, which may even be as high as 10⁶ to 10⁹ copies in the presence of sufficient enzymes and raw materials, but the downside is that selection and design of two primer sequences should be considered, reducing the simplicity of experimental design. In recent years, Murakami et al. designed PG-RCA, i.e., a method for rolling circle amplification where primers can be “manufactured” steadily and spontaneously to maintain amplification during amplification. The method expands the application scope of RCA tremendously. Murakami et al. detected RNAs in combination of the technique with specially designed 3WJ probes and detected tens to hundreds of zeptomoles of RNAs successfully. Again, Wang et al. designed a cyclized DNA nanomachine and achieved the detection of cancer-related genes, K-ras and p53, by means of PG-RCA combined with strand-displacement amplification (SDA), leading to a detection limit of down to 50 pM.

Compared with other exponential RCA techniques, the biggest advantage of PG-RCA is that there is no need to add extra primer, avoiding a complex design of double primers and therefore simplifying an amplification system. Next, such mechanism of primer generation is more able to detect target DNAs which can serve as primers; even under the condition that a primer concentration is very low, signal amplification can still be achieved well.

During PG-RCA, however, exponential RCA cannot proceed until one strand of a double-stranded DNA (dsDNA) is cleaved; amplification will not proceed if both strands of the dsDNA are cleaved. In the prior art, cleavage of only one strand of a dsDNA is achieved by nicking endonucleases. However, nicking endonucleases developed and applied so far are limited by fewer types, easy denaturation and inactivation at high temperature, and strict requirements for reaction temperature, greatly restricting the practical popularization of the technique and partly limiting the application scope thereof. Therefore, it is urgent to develop a more widely used and highly sensitive method for nucleic acid amplification.

### SUMMARY

Based on the defects of nicking endonucleases used in the existing PG-RCA techniques, i.e., fewer types, easy denaturation and inactivation at high temperature, and strict requirements for reaction temperature, the present invention provides a method for restriction endonuclease-mediated primer generation-rolling circle amplification.

To solve the above technical problem, the technical solution adopted by the present invention is as follows:

Disclosed is a method for restriction endonuclease-mediated PG-RCA, including the following steps of:

1) Thiol-modifying a single-stranded DNA (ssDNA) at a restriction site of a restriction endonuclease;

2) Cyclizing the thiol-modified ssDNA to obtain a thiol-modified circular ssDNA;

3) Using the thiol-modified circular ssDNA obtained in step 2) as a template, adding the template into a reaction system of a target DNA primer to be detected, a DNA polymerase with strand displacement, and a restriction endonuclease for multiple cycles of rolling circle amplification, to amplify a molecular signal of target DNA.

The functional principle and process of the method provided by the present invention are described as follows:

First of all, in order to enable a restriction endonuclease to cleave only one strand of a double-stranded DNA (dsDNA), a method for modifying a circular template is therefore used. That is to say, thiol-modification (thiol-modification, i.e., thiophosphate modification, refers to substitution of a non-bridging oxygen atom at alpha position of a phosphodiester bond between nucleotides by a sulfur atom) is conducted at a restriction site of the template to inhibit the cleavage activity of the restriction endonuclease herein, thereby enabling the restriction endonuclease to cleave a strand complementary to a circular DNA only.

As shown in FIG. 1, a circular DNA template is introduced into an amplification system. When a strand of DNA primer to be detected is complementary to a circular DNA in the system, linear RCA will be primed in the presence of a DNA polymerase with strand displacement activity, producing long and continuous single-stranded DNA repeats complementary to the circular DNA. Because there are excessive circular DNAs in the system and the rest of circular DNAs will bind to linear RCA products produced in Cycle 1, restriction sites of restriction endonucleases will continue to be formed; restriction endonucleases in the system recognize and cleave binding sites (where only the strand complementary to the circular DNA can be cleaved) to produce nicks with 3′-end on DNA strands, forming RE-RCA primers to conduct a second cycle of amplification in the presence of DNA polymerases. Similarly, a signal of the original DNA molecule is amplified ultimately after multiple cycles of the foregoing reaction.

Based on the above technical solution, the following improvements can be made on the present invention.

Further, step 2) includes a step of purifying the thiol-modified circular ssDNA with Exo I.

A beneficial effect of using the above further technical solution is that purification is able to remove by-products produced during cyclization and excessive splints so as to purify and enrich circular DNAs.

Further, the reaction system described in step 3) includes 0.01 to 1 ng/μL salmon sperm DNA.

A beneficial effect of using the above further technical solution is that addition of salmon sperm DNA inhibits the conduct of de novo synthesis in the system, avoiding non-specific reactions caused in the presence of the restriction endonuclease and dNTPs.

Further, the cyclization described in step 2) is achieved by T4 DNA Ligase splint-assisted cyclization.

Beneficial effects of using the above further technical solution include high cyclization efficiency and fewer side reactions.

Further, a phosphate group is present at 3′-end of the splint.

A beneficial effect of using the above further technical solution is to prevent the splint from serving as a primer to trigger amplification and to reduce the effect of background noise on detection results.

Further, the length of the single-stranded DNA described in step 1) ranges from 30 to 90 nt.

Beneficial effects of using the above further technical solution are that: shorter DNA strands are too rigid to form circular DNAs; longer DNA strands are prone to make errors during synthesis; DNA strands with a length of 30 to 90 nt enable precise synthesis and easy cyclization, which are most suitable to serve as RCA templates.

Further, the restriction endonuclease may be any one of type II restriction endonucleases, and preferably any one of TspRI, MboI, HinP1I, and AluI.

Further, the DNA polymerase is any one of Phi29 DNA polymerase, Bst DNA polymerase, and Vent(exo-) DNA polymerase.

Further, the template in step 3) has a concentration of 5 to 50 nM and the target DNA primer has a concentration of 1 to 50 nM.

The present invention has the following beneficial effects:

1) Using a restriction endonuclease to prime primer generation and establish a restriction endonuclease-mediated RCA technique, high amplification efficiency can be guaranteed and the application scope of the technique is broadened;

2) With easy operation, nucleic acid amplification can be achieved at a constant temperature; and

3) The present invention can be combined with molecular probe techniques and utilized in visual detection of Pb²⁺.

### BRIEF DESCRIPTION OF DRAWINGS

FIG. 1 illustrates principles of a method for rolling circle amplification provided by the present invention.

FIG. 2 illustrates a design of thiol-modification of a single-stranded DNA template of the present invention.

FIG. 3 shows an electrophoretogram of digested products of dsDNA strands by restriction endonucleases in Example 1. In FIG. 3, Lanes 1 to 3 represent strand markers, Lanes 4 to 6 represent results of TspRI-digested dsDNAs, Lanes 7 to 9 represent results of Mbol-digested dsDNAs, Lanes 10 to 12 represent results of AluI-digested dsDNAs, and Lanes 12 to 15 represent results of HinP1I-digested dsDNAs. In particular, Lanes 4, 7, 10, and 12 represent endonuclease digestion results of unmodified dsDNAs; Lanes 5, 8, 11, and 13 represent endonuclease digestion results of one thiol-modified dsDNA present at a restriction site, respectively; Lanes 6, 9, 12, and 15 represent endonuclease digestion results of three thiol-modified dsDNAs present at a restriction site, respectively.

FIG. 4 shows an electrophoretogram of cyclization of ssDNAs by T4 DNA Ligase in Example 2. In FIG. 4, Lane 1 represents a single strand of T-59sDNA, Lane 2 represents a product of T4 DNA Ligase, and Lane 3 represents an Exo I digested product of ligation product of T4 DNA Ligase.

FIG. 5 shows an electrophoretogram of products of rolling circle amplification at 45 to 70° C. in Example 2. In FIG. 5, Lane L represents D2000Ladder, Lanes 1 to 6 represent products of RE-RCA at 45, 50, 55, 60, 65, and 70° C., respectively.

FIG. 6 shows an electrophoretogram of products of rolling circle amplification at circular template concentrations of 5 to 50 nM in Example 2. In FIG. 6, Lane L represents D2000 Ladder, Lane 1 represents a blank control of the template, and Lanes 2 to 6 represent amplification products at template concentrations of 5, 10, 50, 100, and 200 nM, respectively.

FIG. 7 shows an electrophoretogram of rolling circle amplification in Example 2. In FIG. 7, Lane L represents D2000Ladder. Lanes 1 to 3 represent products of RE-RCA at primer concentrations of 0, 50, and 100 nM, respectively; Lane 4 represents a single strand of T-59s DNA, functioning as an indicator.

FIG. 8 illustrates a design and a detection principle of a Pb²⁺ probe in Example 3.

FIG. 9 shows an electrophoretogram of GR-5Probe digestion at Pb²⁺ concentrations of 0.1 to 20 μM in Example 3. In FIG. 9, Lane 1 represents a Pb²⁺-free blank control, and Lanes 2 to 7 represent results of reactions at Pb²⁺ concentrations of 0.1, 0.5, 1, 5, 10, and 20 μM, respectively.

FIG. 10 illustrates rolling circle amplification at different Pb²⁺ concentrations and fluorescence detection in Example 3.

FIG. 11 illustrates results of amplification curves of blank group versus experimental group (Pb²⁺) at different GR-5Probe concentrations (1, 5, 25, and 50 μM) in Example 3.

FIG. 12 illustrates results of time to reach fluorescence threshold (threshold time, T) of both groups in FIG. 11, which are obtained by calculating and comparing T values of GR-5Probe of the experimental group and the blank group at each concentration.

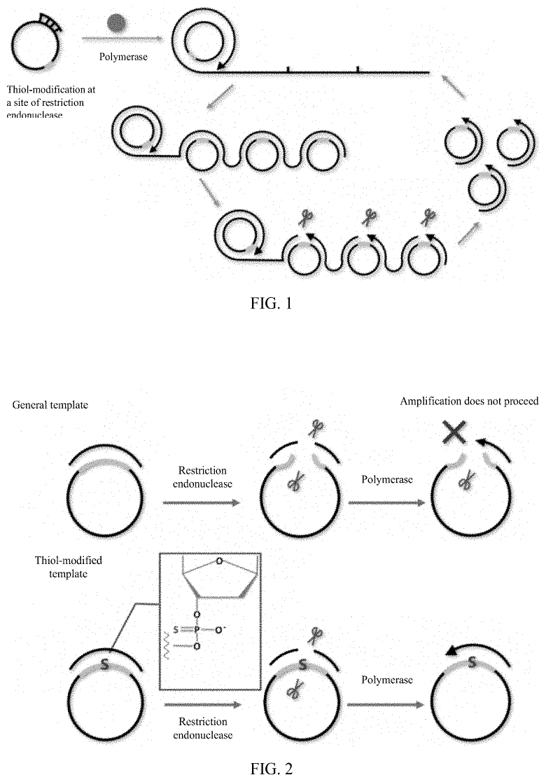

### DETAILED DESCRIPTION

The methods and features of the present invention are described with reference to the following specific examples, and the listed examples only serve to explain the present invention, but are not intended to limit the scope of the present invention.

### EXAMPLE 1

### Effects of the Presence of Thiol-Modification at Recognition Sites of Restriction Endonucleases on Activity of Restriction Endonucleases

1) Absorbance of a single-stranded DNA (ssDNA) was measured at 260 nm using Nanodrop 2000, a sequence concentration was analyzed, and a DNA sequence was diluted to 100 μM.

2) Two DNA sequences were mixed in a 1:1 ratio, denatured at 94° C. for 3 min, and cooled down to 25° C. at a rate of 0.1° C. per second, to synthesize general double strands and dsDNAs with one and three thiol-modifications at restriction sites.

3) dsDNA (1 μM), 0.5 U restriction endonuclease, and 1× Buffer were diluted to 10 μL with ddH₂O. AlUI, MboI, and Hin1I reacted for 3 h at 37° C. and then inactivated for 20 min at 65° C.; TspRI reacted for 3 h at 65° C.

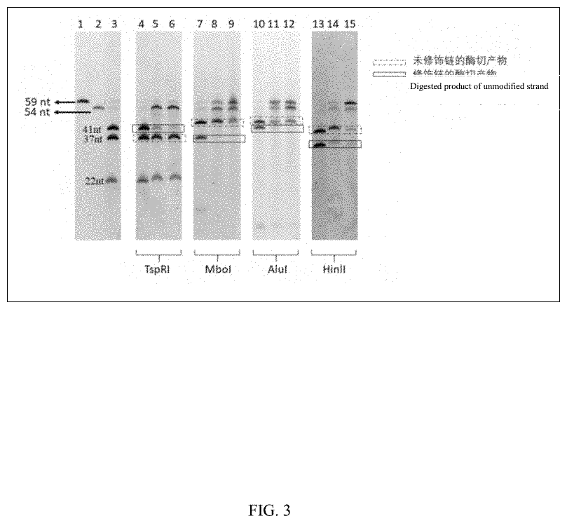

4) Amplification products analysis by gel electrophoresis. Amplification products were analyzed by denaturing polyacrylamide gel electrophoresis (PAGE) with 12% formamide (voltage, 350 V; electrophoresis time 3 h). Electrophoresis results were observed by SYBR Green II staining, and products were analyzed quantitatively in Image Lab software depending on the band brightness. As shown in FIG. 3, thiol-modification has a certain hindering effect on restriction endonucleases. When thiol-modification is present in the sequence, restriction endonuclease(s) will not (or hardly) have the modified sequence cleaved, but cleave the other unmodified strand normally; on the other hand, for certain hindering effect on restriction endonucleases, a strand with three thiol-modifications is better than that with one thiol-modification. Subsequently, for example, TspRI and Vent (exo-) DNA polymerase were used to trigger restriction endonuclease-mediated primer generation-rolling circle amplification.

### EXAMPLE 2

### A Method for Restriction Endonuclease-Mediated Primer Generation-Rolling Circle Amplification (RCA)

Raw material of single-stranded DNA (ssDNA):
T-59:
(SEQ ID NO. 1)
5′-TCTTCCTCAGCGAAGCAGTGTA*TCTGAATGCCAGTCTGATAAGCCC
ACGTGACATCCTG-3′
(* represents a position of thiol-modification;
underline represents a TspRI restriction site).
Splint:
(SEQ ID NO. 2)
5′-p-CGCTGACAGGAT p-3′
(p represents where a phosphate group is present)
Primer:
(SEQ ID NO. 3)
5′-CTGACGCTGACAGGATGTCA-3′

1) Absorbance of a single-stranded DNA (ssDNA) was measured at 260 nm using Nanodrop 2000, a sequence concentration was analyzed, and T-59 was diluted to 100 μM.

2) The ssDNA was subjected to thiophosphate modification (i.e., thiol-modification). System: ssDNA 10 μM; T4 PNK Kinase 5 U; T4 PNK Kinase Buffer A 1×; ATP 1 mM; diluted to 20 μL with ddH₂O. The system reacted for 6 h at 37° C., enzymes were inactivated for 10 min at 70° C., and then the treated DNA was transferred to and stored in a refrigerator at −20° C. for use.

3) Cyclization: 1 μM thiol-modified template ssDNA, 2 μM splint, and 1× T4 DNA Ligase buffer were mixed and diluted to 19 μL with water; the mixture was placed in a preheated PCR system, denatured at 94° C. for 3 min, and cooled down to 25° C. at a rate of 0.1° C. per second; finally, 2.5 U DNA Ligase was added. The mixture reacted for 4 h at 25° C., followed by inactivation for 20 min at 65° C.

4) Purification: 1 μM ligation product, 20 U Exo I and 1× Exo I Buffer were mixed, followed by reaction for 1 h at 37° C. and inactivation for 20 min at 80° C.

5) Rolling circle amplification (RCA): 5 to 50 nM circular DNA template, 50 nM primer, 1× ThermoPol Reaction Buffer (20 mM Tris-HCl, 10 mM (N₄)₂SO₄, 10 mM KCl, 2 mM MgSO₄, 0.1% Triton® X-100, pH 8.8), 400 μM dNTPs, 4 mM MgSO₄, 1 U TspRI, 10 ng of salmon sperm DNA, and 4 U Vent (exo-) DNA polymerase were mixed, and diluted to 10 μL with ddH₂O. Reaction temperature was 45 to 70° C. The mixture reacted for 30 min at a constant temperature to obtain amplification products.

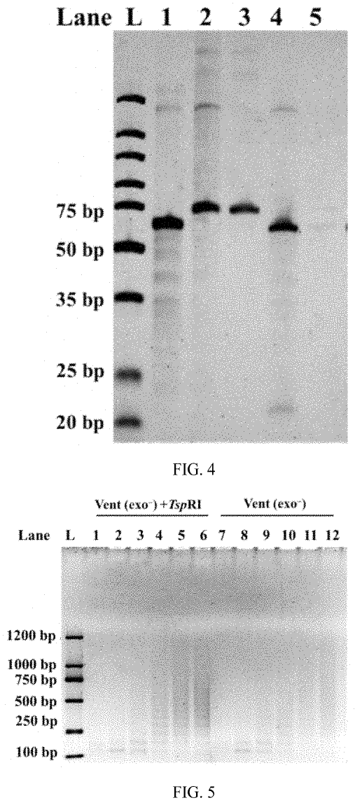

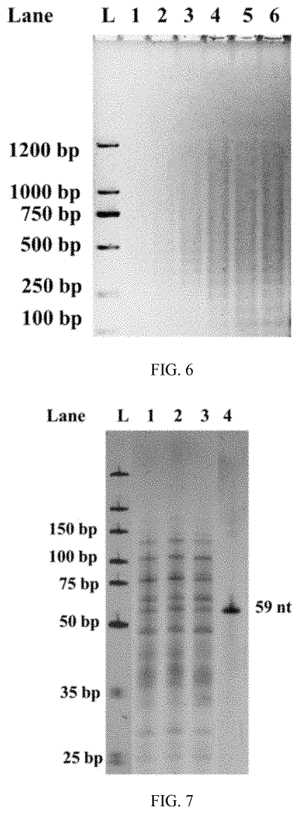

6) Amplification product analysis by gel electrophoresis. Amplification products were analyzed by 8% denaturing polyacrylamide gel electrophoresis (PAGE) (voltage, 300 V; electrophoresis time 2 h). Electrophoresis results were observed by SYBR Green II staining, and products were analyzed quantitatively in Image Lab software depending on the band brightness. Then, products were electrophoresed on 1% Agarose for 70 min at 120 V, stained with Gene Green, and qualitatively analyzed depending on the band brightness. As shown in FIG. 4, a circular template required by the experiment can be prepared using T4 DNA Ligase, and those excessive nucleic acids can be eliminated by Exo I, functioning as a template purifier. FIG. 5 shows a gel electrophoretogram of amplification products obtained at a template concentration of 50 nM and at a reaction temperature of 45 to 70° C. As shown in FIG. 5, amplification products increase gradually with increasing temperature, and peak at 65° C. FIG. 6 shows a gel electrophoretogram of amplification products obtained at a template concentration of 5 to 50 nM and at a reaction temperature of 65° C. As shown in FIG. 6, as the template concentration increases, excessive templates bind better to linear RCA products; further, products increase gradually as more primers are produced; the amount of products peaks when the template concentration reaches 50 nM. As shown in FIG. 7, primer generation provided by the present invention can electrophoretically detect 1 nM DNA primer, exhibiting a good amplification level.

### EXAMPLE 3

First of all, a Pb²⁺ probe with specific recognition was designed. GR-5DNAzyme served as a main body of the probe and a functional domain to recognize Pb²⁺, and GR-5Probe was designed on the basis of GR-5DNAzyme. In the absence of Pb²⁺, the probe per se could maintain a steady secondary structure; in the presence of Pb²⁺, the probe released digested products as many as possible to form a free single strand after endonuclease digestion. Noteworthily, a circular ring of 5′-CGAAGC-3′ designed on the probe was a 3 bp small hairpin, which had a melting point (Tₘ) of up to 78.3° C. Ligation of GR-5E and substrate GR-5S with the small hairpin could enable the probe to maintain a relatively steady secondary structure; also, a sequence of binding domain with approximately 50% of GC was designed. A binding domain proximal to one end of the hairpin had a length of 12 bp, while that distal to the other end of the hairpin was 20 bp (10 nt+12 nt) double strands with cohesive ends, where two bases (TT) extruded at 3′-end. The purpose of such design was to enable better binding of the single strand of the digested product released after probe digestion to the circular template. The total length of the probe was 89 nt because bases of the functional domain are constant. The probe recognized Pb²⁺ and enzymatically digested to produce S, a 25 nt single strand with one rA base. S, as a primer, could bind to the circular template in the system, trigger restriction endonuclease-mediated primer generation-rolling circle amplification, and finally realize the signal amplification of action of Pb²⁺.

The following is an example of Pb²⁺ detection.

Raw material of DNA:
T-79:
(SEQ ID NO. 4)
5′-TTAGTCTCCGAGCAGTGTA*TGAGGTTTCAATTGCCCACGGTACATC
CTGTCTTCCTTTAAAGAACTCCACCA-3′
(* represents a position of thiol-modification;
underline represents a TspRI restriction site).
GR-5probe:
(SEQ ID NO. 5)
5′-GCCCACGAGACATCCTGTCTGAAGTAGCGCCGCCGTACACGCCGTGA
CGAAGTCACGGCGTGTrAGGAAGACAGGATGTCCCGTGGGCAA-3′.
Primer:
(SEQ ID NO. 6)
5′-AGGAAGACAGGATGTCCCGTGGGCAA-3′

1) Absorbance of a T-59 sequence was measured at 260 nm using Nanodrop 2000, a sequence concentration was analyzed, and T-59 was diluted to 100 μM.

2) A single-stranded DNA (ssDNA) ordered was thiol-modified. System: ssDNA 10 μM; T4 PNK Kinase 5 U; T4 PNK Kinase Buffer A 1×; ATP 1 mM; diluted to 20 μL with ddH₂O. The system reacted for 6 h at 37° C., enzymes were inactivated for 20 min at 65° C., and then the phosphorylated DNA was transferred to and stored in a refrigerator at −20° C. for use.

3) Cyclization: 1 μM thiol-modified template ssDNA, 2 μM splint, and 1× T4 DNA Ligase buffer were mixed and diluted to 19 μL with water; the mixture was placed in a preheated PCR system, denatured at 94° C. for 3 min, and cooled down to 25° C. at a rate of 0.1° C. per second; finally, 2.5 U DNA Ligase was added. The mixture reacted for 4 h at 25° C., followed by inactivation for 20 min at 65° C.

4) Purification: 1 μM ligation product, 20 U Exo I and 1× Exo I Buffer were mixed, followed by reaction for 1 h at 37° C. and inactivation for 20 min at 80° C.

5) GR-5Probe digestion: 400 nM GR-5Probe, 50 mM HEPES/Tris-HCl/PBS, 4 mM MgCl₂, and 0.1 to 20 μM Pb(NO₃)₂ were mixed and diluted to 5 μL with water.

6) Rolling circle amplification (RCA): 5 μL of digested product of GR-5Probe, 100 nM circular DNA template, 1× ThermoPol Reaction Buffer, 800 μM dNTPs, 4 mM MgSO₄, 2 U TspRI, 2 ng of salmon sperm DNA, and 8 U Bst DNA polymerase were mixed, and diluted to 10 μL with ddH₂O. Reaction temperature was 65° C. The mixture reacted for 30 min at a constant temperature.

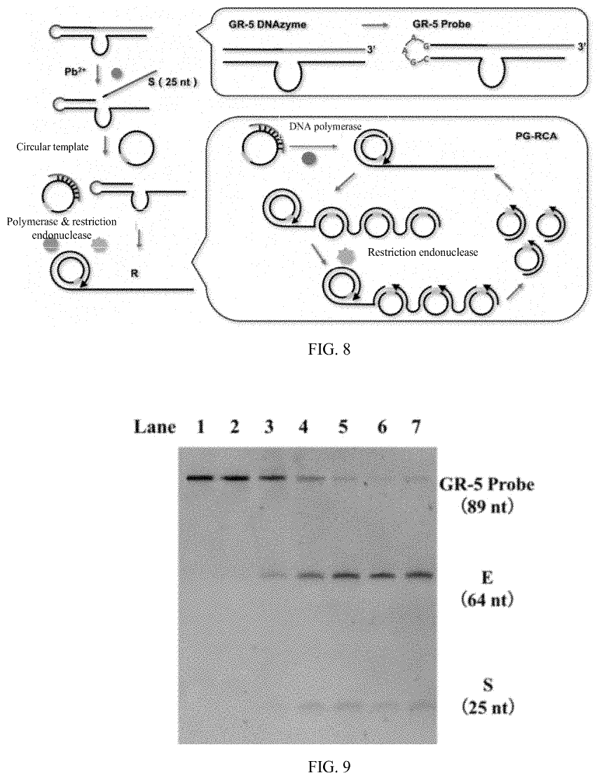

7) Products analysis by gel electrophoresis. Products were analyzed by 8% denaturing polyacrylamide gel electrophoresis (PAGE) (voltage, 300 V; electrophoresis time 2 h). Electrophoresis results were observed by SYBR Green II staining, and products were analyzed quantitatively in Image Lab software depending on the band brightness. Then, products were electrophoresed on 1% Agarose for 70 min at 120 V, stained with Gene Green, and qualitatively analyzed depending on the band brightness. As shown in FIG. 8, GR-5Probe can recognize Pb²⁺, and cleavage efficiency increases with increasing Pb²⁺ concentration, functioning as a good indicator; the probe is feasible for Pb²⁺ detection. As shown in FIG. 9, as Pb²⁺ concentration increases, fluorescence intensity grows rapidly, and RE-RCA enters the exponential growth phase more quickly. This suggests that, with a high concentration of Pb²⁺, higher cleavage efficiency of the probe results in more digested products, thereby accelerating RE-RCA efficiency. Compared with blank group, it concludes that Pb²⁺ can be detected as low as 1 nM using the method.

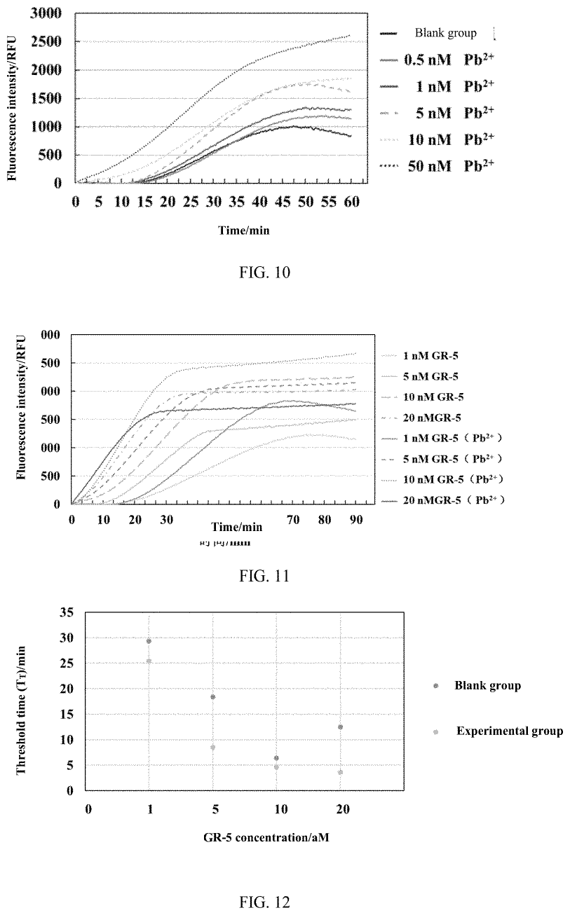

8) Fluorescence detection: 5 μL of digested product of GR-5Probe, 100 nM circular DNA template, 1× ThermoPol Reaction Buffer, 800 μM dNTPs, 4 mM MgSO₄, 2 U TspRI, 2 ng of salmon sperm DNA, 8 U Vent (exo-) DNA polymerase, and 2× Eva Green were mixed, and diluted to 10 μL with ddH₂O. Reaction temperature was 65° C. The mixture reacted for 30 min at a constant temperature. As shown in FIG. 10, at each GR-5Probe concentration, trends of amplification curves of blank and experimental groups appear basically the same; the fluorescence intensity gradually grows exponentially over time and finally becomes steady. FIG. 11 illustrates the time to reach fluorescence threshold (threshold time, T) for each group in FIG. 10. Results show that there is a maximal difference in T (ΔT=9.92 min) between blank and experimental groups when GR-5Probe is 5 nM in the system, suggesting a minor effect of background signal on detection. Therefore, it can be confirmed that a 1:10 concentration ratio of GR-5Probe in the detection system to template is optimal.

The above merely describes preferred examples of the present invention, but is not intended to limit the present invention. Any modifications, equivalent replacements or improvements made within the spirit and principle of the present invention shall fall within the protection scope of the present invention.

1. A method for restriction endonuclease-mediated primer generation-rolling circle amplification, comprising the following steps of:
1) thiol-modifying a single-stranded DNA (ssDNA) at a restriction site of a restriction endonuclease;
2) cyclizing the thiol-modified ssDNA to obtain a thiol-modified circular ssDNA;
3) using the thiol-modified circular ssDNA obtained in step 2) as a template, adding the template into a reaction system of a target DNA primer to be detected, a DNA polymerase with strand displacement, and a restriction endonuclease for multiple cycles of rolling circle amplification, to amplify a molecular signal of target DNA.

2. The method for primer generation-rolling circle amplification according to claim 1, wherein step 2) further comprises a step of purifying the thiol-modified circular ssDNA with Exo I.

3. The method for primer generation-rolling circle amplification according to claim 1, wherein the reaction system described in step 3) further comprises 0.01 to 1 ng/μL salmon sperm DNA.

4. The method for primer generation-rolling circle amplification according to claim 3, wherein the cyclization described in step 2) is achieved by T4 DNA Ligase splint-assisted cyclization.

5. The method for primer generation-rolling circle amplification according to claim 4, wherein a phosphate group is present at 3′-end of the splint.

6. (Curently Amended) The method for primer generation-rolling circle amplification according to claim 1, wherein the restriction endonuclease may be any one of type II restriction endonucleases.

7. The method for primer generation-rolling circle amplification according to claim 6, wherein the restriction endonuclease may be any one of TspRI, MboI, HinP1I, and AluI.

8. The method for primer generation-rolling circle amplification according to claim 1, wherein the DNA polymerase is any one of Phi29 DNA polymerase, Bst DNA polymerase, and Vent(exo-) DNA polymerase.

9. The method for primer generation-rolling circle amplification according to claim 1, wherein the length of the single-stranded DNA described in step 1) ranges from 30 to 90 nt.

10. The method for primer generation-rolling circle amplification according to claim 1, wherein the template described in step 3) has a concentration of 5 to 50 nM and the target DNA primer has a concentration of 1 to 50 nM.

11. The method for primer generation-rolling circle amplification according to claim 2, wherein the restriction endonuclease may be any one of type II restriction endonucleases.

12. The method for primer generation-rolling circle amplification according to claim 4, wherein the restriction endonuclease may be any one of type II restriction endonucleases.

13. The method for primer generation-rolling circle amplification according to claim 11, wherein the restriction endonuclease may be any one of TspRI, MboI, HinP1I, and AluI.

14. The method for primer generation-rolling circle amplification according to claim 12, wherein the restriction endonuclease may be any one of TspRI, MboI, HinP1I, and AluI.

15. The method for primer generation-rolling circle amplification according to claim 2, wherein the DNA polymerase is any one of Phi29 DNA polymerase, Bst DNA polymerase, and Vent(exo-) DNA polymerase.

16. The method for primer generation-rolling circle amplification according to claim 4, wherein the DNA polymerase is any one of Phi29 DNA polymerase, Bst DNA polymerase, and Vent(exo-) DNA polymerase.

17. The method for primer generation-rolling circle amplification according to claim 2, wherein the length of the single-stranded DNA described in step 1) ranges from 30 to 90 nt.

18. The method for primer generation-rolling circle amplification according to claim 4, wherein the length of the single-stranded DNA described in step 1) ranges from 30 to 90 nt.

19. The method for primer generation-rolling circle amplification according to claim 2, wherein the template described in step 3) has a concentration of 5 to 50 nM and the target DNA primer has a concentration of 1 to 50 nM.

20. The method for primer generation-rolling circle amplification according to claim 4, wherein the template described in step 3) has a concentration of 5 to 50 nM and the target DNA primer has a concentration of 1 to 50 nM.


SEQUENCE LISTING (6 sequences)

SEQ ID NO: 1 - DNA, 59 bp, artificial sequence
  T-59
tcttcctcagcgaagcagtgtatctgaatgccagtctgataagcccacgtgacatcctg

SEQ ID NO: 2 - DNA, 12 bp, artificial sequence
  Splint
cgctgacaggat

SEQ ID NO: 3 - DNA, 20 bp, artificial sequence
  Primer
ctgacgctgacaggatgtca

SEQ ID NO: 4 - DNA, 72 bp, artificial sequence
  T-79
ttagtctccgagcagtgtatgaggtttcaattgcccacggtacatcctgtcttcctttaaagaactccacca

SEQ ID NO: 5 - DNA, 90 bp, artificial sequence
  GR-5probe
gcccacgagacatcctgtctgaagtagcgccgccgtacacgccgtgacgaagtcacggcgtgtraggaagacaggatgtcccgtgggcaa

SEQ ID NO: 6 - DNA, 26 bp, artificial sequence
  Primer
aggaagacaggatgtcccgtgggcaa